(19) United States
(12) Patent Application Publication
(10) Pub. No.: US 2023/0278209 A1
(43) Pub. Date: Sep. 7, 2023

(54) SYSTEMS AND METHODS FOR CONTROLLING A ROBOTIC ARM

(71) Applicant: Mazor Robotics Ltd., Caesarea (IL)

(72) Inventors: Ido Zucker, Tel Aviv (IL); Yonatan Ushpizin, Glil Yam (IL); Avraham Turgeman, Beer Yaakov (IL); Eliyahu Zehavi, Tel Aviv (IL); Adi Ess, Ramat Gan (IL)

(21) Appl. No.: 17/590,961

(22) Filed: Feb. 2, 2022

(51) Int. Cl. B25J 9/16

(52) U.S. Cl. CPC B25J 9/1664; B25J 9/163

(57) ABSTRACT

Systems and methods for controlling a robotic arm are provided. A breathing pattern of a patient may be controlled and a sample of the breathing pattern may be obtained. A pattern of movement of an anatomical element based on the sample may be determined. A trajectory of a robotic arm may be adjusted based on the pattern of movement.

### BACKGROUND

The present disclosure is generally directed to controlling a robotic arm, and relates more particularly to controlling a robotic arm using a pattern of movement of an anatomical element determined based on a sample of a patient breathing pattern.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A system for controlling a robotic arm according to at least one embodiment of the present disclosure comprises a robotic arm; a processor; and a memory storing data for processing by the processor, the data, when processed, causes the processor to: control a breathing pattern of a patient; obtain a sample of the breathing pattern; determine a pattern of movement of an anatomical element based on the sample; and adjust a trajectory of the robotic arm based on the pattern of movement.

Any of the aspects herein, further comprising: a marker disposed on the anatomical element and a navigation system configured to track the marker, wherein the sample is obtained from the navigation system tracking a movement of the marker during a time period.

Any of the aspects herein, wherein the marker comprises at least one of an optical marker, an infrared light emitting diode, an electromagnetic marker, and an inertial measurement unit tracker.

Any of the aspects herein, wherein determining the pattern of movement of the anatomical element comprises determining a maximum height and a minimum height of the movement of the marker and a number of movements of the marker per the time period.

Any of the aspects herein, further comprising: a ventilator configured to control the breathing pattern of the patient.

Any of the aspects herein, wherein the sample is obtained from the ventilator and the sample is based on the breathing pattern controlled by the ventilator.

Any of the aspects herein, wherein the sample is a waveform representing the breathing pattern, and wherein determining the pattern of movement comprises determining at least one of a crest, a trough, an amplitude, and a period of the waveform.

Any of the aspects herein, further comprising: a sensor coupled to the robotic arm, the sensor configured to provide pose information of the robotic arm, wherein the robotic arm is coupled to the anatomical element, and wherein the sample is obtained from the sensor, the sample comprising a plurality of poses of the robotic arm for a time period.

Any of the aspects herein, wherein the plurality of poses corresponds to a movement of the anatomical element, and wherein determining the pattern of movement of the anatomical element comprises determining a maximum height and a minimum height of the movement and a number of movements of the robotic arm per the time period.

Any of the aspects herein, wherein the sample is a first sample and the pattern of movement is a first pattern of movement, and the memory stores further data for processing by the processor that, when processed, causes the processor to: obtain a second sample of the breathing pattern; determine a second pattern of movement of an anatomical element based on the sample; and adjust the trajectory of the robotic arm based on the second pattern of movement when the second pattern of movement does not match the first pattern of movement.

Any of the aspects herein, wherein the trajectory is adjusted in at least one coordinate direction.

Any of the aspects herein, wherein the anatomical element comprises one or more vertebrae.

A system for controlling a robotic arm according to at least one embodiment of the present disclosure comprises a robotic arm coupled to an anatomical element; a sensor coupled to the robotic arm, the sensor configured to provide pose information of the robotic arm; a processor; and a memory storing data for processing by the processor, the data, when processed, causes the processor to: obtain a sample of a patient breathing pattern comprising a plurality of poses for a time period from the sensor; determine a pattern of movement of the anatomical element based on the sample; and adjust a trajectory of the robotic arm based on the pattern of movement.

Any of the aspects herein, wherein the plurality of poses corresponds to a movement of the anatomical element, and wherein determining the pattern of movement of the anatomical element comprises determining a maximum height and a minimum height of the movement and a number of movements of the robotic arm per the time period.

Any of the aspects herein, further comprising: a ventilator configured to control the breathing pattern of the patient.

Any of the aspects herein, wherein the trajectory is adjusted in at least one coordinate direction.

A system for controlling a robotic arm according to at least one embodiment of the present disclosure comprises a marker disposed on an anatomical element; a navigation system configured to track the marker; a processor; and a memory storing data for processing by the processor, the data, when processed, causes the processor to: track the marker; obtain a sample of a patient breathing pattern comprising a plurality of poses of the marker for a time period from the navigation system; determine a pattern of movement of an anatomical element based on the sample; and adjust a trajectory of the robotic arm based on the pattern of movement.

Any of the aspects herein, wherein the plurality of poses correspondent to a movement of the anatomical element, and wherein determining the pattern of movement of the anatomical element comprises determining a maximum height and a minimum height of the movement and a number of movements of the marker per the time period.

Any of the aspects herein, further comprising: a ventilator configured to control the breathing pattern of the patient.

Any of the aspects herein, wherein the marker comprises at least one of an optical marker, an infrared light emitting diode, an electromagnetic marker, and an inertial measurement unit tracker.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a flowchart according to at least one embodiment of the present disclosure; and

FIG. 3 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In a surgical operation using a robotic surgical system, a user such as a surgeon may plan a surgical procedure comprising, for example, screw planning, drilling bone milling, soft tissue extraction, etc. Prior to a start of the surgical procedure, a patient anatomy may be registered to the system. During the operation the patient may move in reference to the system. In order to track a patient movement, a reference frame marker may be placed on the patient anatomy. The tracked marker may move with the patient and the robotic system may react to the patient movement. In such conventional examples, the system may recognize the patient movement and react to such movements. Some of the patient movement may be repeatable, such as the patient breathing. For example, in the operating room, the patient is sedated and the breathing may be induced by the anesthesiologist.

In at least one embodiment of the present disclosure, a robotic system may sample the patient movement due to the patient breathing and the robotic system may predicate its movement in anticipation of the patient breathing. In such embodiments, a lag time between the patient movement and the robotic reaction may be reduced. In some embodiments, a marker may be placed on the patient anatomy and tracked to obtain a sample of the patient movement. The marker may be optical, metal, or electronic. In other embodiments, the system may connect to the anesthesia device to track the patient movement. In some embodiments, repeatable movement may be acute due to movement that is performed directly by the robotic system. In some embodiments, the patient movement may be sampled and a position that will minimize an error or provide a safe position for a surgical instrument or tool may be determined. In some embodiments, e.g., for pedicle screws, the system may adjust movement of a surgical instrument or tool laterally. In such embodiments, the system may not adjust movement of the surgical instrument or tool medially.

Embodiments of the present disclosure provide technical solutions to one or more of the problems of (1) adjusting a trajectory of a robotic arm based on movement of a patient induced by a patient breathing pattern, (2) reducing or eliminating lag between a movement of a patient induced by a patient breathing pattern and a reaction of a robotic arm, (3) increasing patient safety during a surgical operation.

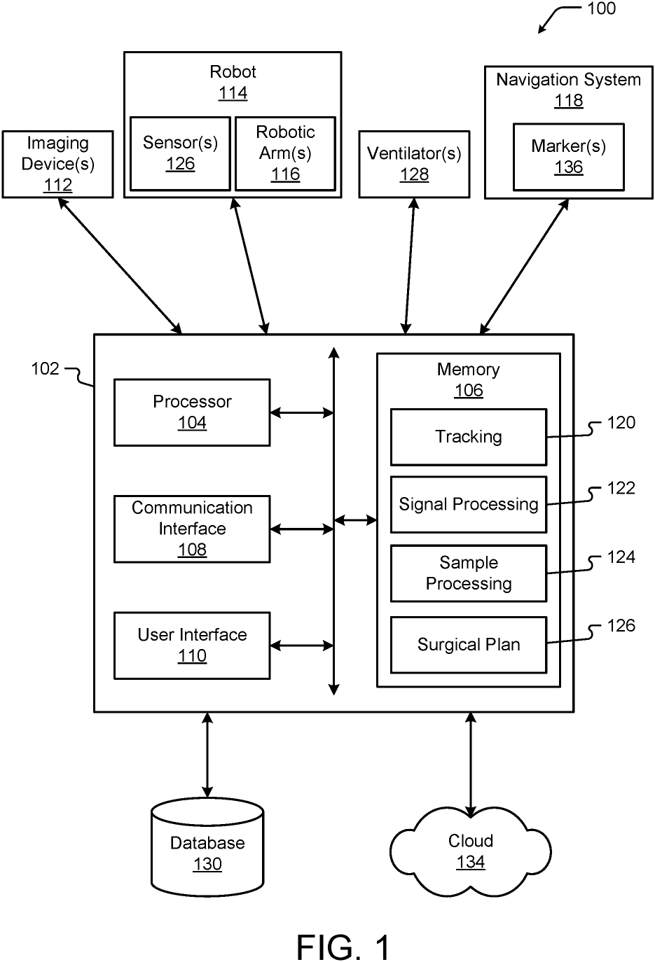

Turning first to FIG. 1, a block diagram of a system 100 according to at least one embodiment of the present disclosure is shown. The system 100 may be used to control a robotic arm, e.g., control, pose, and/or otherwise manipulate a surgical arm, and/or surgical tools attached thereto and/or carry out one or more other aspects of one or more of the methods disclosed herein. The system 100 comprises a computing device 102, one or more imaging devices 112, a robot 114, a ventilator 128, a navigation system 118, a database 130, and/or a cloud or other network 134. Systems according to other embodiments of the present disclosure may comprise more or fewer components than the system 100. For example, the system 100 may not include the imaging device 112, the robot 114, the ventilator 128, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the ventilator 128, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 200, 300 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable tracking 120, signal processing 122, and/or sample processing 124.

The tracking 120 enables the processor 104 or a processor of the navigation system 118 to track the marker 136. The marker 136 may comprise one or more markers disposed on, for example, the patient, a surgical tool, a surgical instrument, or any component in the operating area. The tracking 120 may, for example, enable the processor 104 to process image data (which may be received from, for example, an imaging device such as the imaging device 112 or an imaging device of a navigation system such as the navigation system 118) or sensor data (which may be received from, for example, a sensor such as the sensor 126) for the purpose of identifying the marker 136 and/or obtaining identifying information about the marker 136 from the image data or the sensor data. The information may comprise, for example, a plurality of poses of the marker 136. The plurality of poses may correspond to a patient breathing pattern and a sample of the plurality of poses may be taken for a time period. The information obtained from the tracking 120 may enable the navigation system 118 to, for example, track the marker 136 and obtain a sample of a patient breathing pattern. The sample obtained from the tracking 120 may also be processed by a processor such as the processor 104 to determine a pattern of movement, as described below.

The signal processing 122 enables the processor 104 to process a signal (received from, for example, a ventilator such as the ventilator 128) for the purpose of, for example, converting the signal to a waveform representing a patient breathing pattern. A sample of the waveform may be taken for a time period. The sample obtained from the signal processing 122 may be processed by a processor such as the processor 104 to determine a pattern of movement, as described below.

The sample processing 124 enables the processor 104 to process a sample of a patient breathing pattern (received from for example, the tracking 120 and/or the signal processing 122) for the purpose of, for example, determining a pattern of movement. The pattern of movement may be a pattern of movement of the entire patient due to the patient breathing. In other instances, the pattern of movement may be of an anatomical element due to the patient breathing. The anatomical element may be, for example, a vertebra, a plurality of vertebrae, etc. The pattern of movement obtained from the sample processing 124 may enable the robot 114 and/or the robotic arm 116 to adjust its trajectory or movement accordingly.

Content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the ventilator 128, the database 130, and/or the cloud 134.

The memory 106 may also store a surgical plan 126. The surgical plan 126 may comprise, for example, one or more steps for performing a surgical procedure. In some embodiments, the surgical procedure may be a spinal procedure (e.g., a spinal alignment, installing implants, osteotomy, fusion, and/or any other spinal procedure) to correct a spinal deformity. For example, the surgical plan 126 may comprise one or more surgical steps for moving a plurality of vertebrae to a predetermined alignment. In another example, the surgical plan 126 may comprise one or more surgical steps and one or more trajectories for drilling a plurality of vertebrae. The surgical plan 126 may also be stored in the database 130.

The computing device 102 may also comprise a communication interface 108. The communication interface 108 may be used for receiving image data or other information from an external source (such as the imaging device 112, the robot 114, the ventilator 128, the navigation system 118, the database 130, the cloud 134, and/or any other system or component not part of the system 100), and/or for transmitting instructions, images, or other information to an external system or device (e.g., another computing device 102, the imaging device 112, the robot 114, the ventilator 128, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.1 1a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or marker(s) 136 to yield image data or sensor data (e.g., image data depicting or corresponding to a bone, veins, tissue, etc. or image data or sensor data corresponding to one or more markers 136). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof, or the marker 136. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient and/or the marker 136. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robot 114 may comprise one or more sensors 126. The sensor 126 may be a position sensor, a proximity sensor, a magnetometer, or an accelerometer. In some embodiments, the sensor 126 may be a linear encoder, a rotary encoder, or an incremental encoder. In still other embodiments, the sensor 126 may be an imaging sensor. Other types of sensors may also be used as the sensor 126. The one or more sensors 126 may be positioned, for example, on the robotic arm 116, a patient anatomy, any component of the system 100, or any component outside of the system 100.

Data from the sensor(s) 126 may be provided to a processor of the robot 114, to the processor 104 of the computing device 102, and/or to a processor of the navigation system 118. The data may be used to calculate a position in space of the robotic arm 116 (or any component on which the sensor 126 is positioned on or integrated with) relative to one or more coordinate systems (e.g., a navigation coordinate system, a patient coordinate system, etc.). The calculation may be based not just on data received from the sensor(s) 126, but also on data or information (such as, for example, physical dimensions) about, for example, the robot 114 or a portion thereof, or any other relevant object, which data or information may be stored, for example, in a memory 106 of a computing device 102 or in any other memory.

In some embodiments, marker(s) 136 (e.g., navigation markers) may be placed on the robot 114 (including, e.g., on the robotic arm 116), the imaging device 112, a patient, or any other object in the surgical space. The marker 136 may comprise one or more active markers, one or more passive markers, or a combination of active and passive markers. The marker 136 may be, for example, light emitting diodes, infrared light emitting diodes, reflective markers, optical markers, electromagnetic markers, inertial measurement unit trackers, or the like. The marker 136 may be tracked by the navigation system 118, and information from the tracking (e.g., a sample of a patient breathing comprising a plurality of poses of the markers 136) may be used by a processor such as the processor 104 or a processor of the robot 114 and/or by an operator of the system 100 or any component thereof. In some embodiments, the navigation system 118 can be used to track other components of the system (e.g., imaging device 112).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The ventilator 128 may be configured to control a breathing pattern of a patient such that the breathing pattern may remain substantially fixed and continuous during use of the ventilator 128. The breathing pattern may be determined preoperatively or, in other instances, near or at a beginning of a surgical procedure. The breathing pattern may be based on one or more factors of the patient such as age, health, etc. The ventilator 128 may be configured to mechanically pump air (which may include, for example, oxygen) into a patient and control a number of breaths per minute of the patient. The breaths per minute may define the breathing pattern. During use, the ventilator 128 may provide a signal that may be processed by the processor 104 using the signal processing 122 to convert the signal to a waveform. A sample of the breathing pattern may comprise a sample of the waveform for a time period. As previously described, the processor 104 may process the sample using, for example, the sample processing 124 to determine a pattern of movement of a patient or of one or more anatomical elements of the patient.

The database 130 may store information that correlates one coordinate system to another (e.g., one or more robotic coordinate systems to a patient coordinate system and/or to a navigation coordinate system). The database 130 may additionally or alternatively store, for example, one or more surgical plans such as the surgical plan 126 (including, for example, pose information about a target and/or image information about a patient’s anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 200, 300 described herein. The system 100 or similar systems may also be used for other purposes.

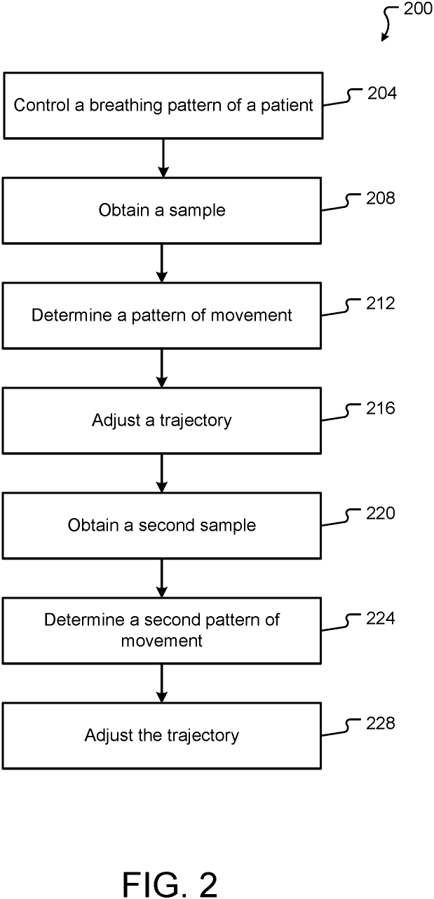

FIG. 2 depicts a method 200 that may be used, for example, for controlling a robotic arm.

The method 200 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 200. The at least one processor may perform the method 200 by executing elements stored in a memory such as the memory 106. The elements stored in the memory and executed by the processor may cause the processor to execute one or more steps of a function as shown in method 200. One or more portions of a method 200 may be performed by the processor executing any of the contents of memory, such as a tracking 120, a signal processing 122, and/or a sample processing 124.

The method 200 comprises controlling a breathing pattern of a patient (step 204). In some embodiments, the breathing pattern of the patient may be controlled by a ventilator such as the ventilator 128. The ventilator may be configured to control the breathing pattern of the patient such that the breathing pattern may remain substantially fixed and continuous during use of the ventilator. In other words, the ventilator may be configured to control a number of breaths per minute of the patient by mechanically pumping air (which may contain, for example, oxygen) into the patient. In this way, the ventilator may create a patient motion that is substantially consistent, controlled, and/or predictable.

The method 200 also comprises obtaining a sample (step 208). In some embodiments, the sample is obtained from the ventilator. More specifically, a signal from the ventilator may be received and processed by a processor such as the processor 104 using a signal processing such as the signal processing 122 to convert the signal to a waveform. A sample of the waveform may be obtained for a time period. The time period may be, for example, about five seconds. In other embodiments, the time period may be less than or greater than five seconds.

In other embodiments, a marker such as the marker 136 may be disposed on an anatomical element (which may be, in some instances, a target anatomical element on which a surgical procedure is to be performed) and a navigation system such as the navigation system 118 may be configured to track the marker (as described in step 304 of method 300 below). In such embodiments, the sample may be obtained from the navigation system tracking a movement of the marker during the time period. The sample may comprise a plurality of poses of the marker for the time period corresponding to a movement of the anatomical element induced by the breathing pattern.

In still other embodiments, a sensor such as the sensor 126 may be coupled to or integrated with a robotic arm such as the robotic arm 116. The sensor may be configured to provide pose information of the robotic arm and the robotic arm may be coupled to or contacting the anatomical element. In such embodiments, the sample may be obtained from the sensor and may comprise a plurality of poses of the robotic arm for the time period. The plurality of poses of the robotic arm may correspond to a movement of the anatomical element induced by the breathing pattern.

The method 200 also comprises determining a pattern of movement (step 212). The pattern of movement may be determined based on the sample received in, for example, the step 208. In some embodiments, the sample may be processed by a processor such as the processor 104 using a sample processing such as the sample processing 124 to determine a pattern of movement based on the sample. In some embodiments, determining the pattern of movement may be based on one or more factors such as, for example, patient data such as an age, height, weight, etc. of the patient. The pattern of movement may be a pattern of movement of a patient and/or of one or more anatomical elements of the patient. The pattern of movement for the one or more anatomical elements may vary based on the target anatomical element as anatomical elements in one area of the patient may move more or less than anatomical elements in another area of the patient. For example, an anatomical element in the thoracic spine may move more than an anatomical element in the lumbar spine. In such examples, the pattern of movement of an anatomical element in the thoracic spine may have a larger distance of movement and/or a greater number of movements per time period than an anatomical element in the lumbar spine.

In embodiments where the sample comprises a waveform, determining the pattern of movement may comprise determining at least one of a crest, a trough, an amplitude, and/or a period of the waveform. In embodiments where the sample comprises a plurality of poses of the marker, determining the pattern of movement of the anatomical element may comprise determining a maximum height and a minimum height of the movement of the marker and a number of movements of the marker per the time period. In embodiments where the sample comprises a plurality of poses of the robotic arm, determining the pattern of movement of the anatomical element may comprise determining a maximum height and a minimum height of the movement and a number of movements of the robotic arm per the time period.

The method 200 also comprises adjusting a trajectory (step 216). The trajectory may correspond to a trajectory of the robotic arm. In some embodiments, a portion of the trajectory may be adjusted. In other embodiments, the entire trajectory may be adjusted. In some embodiments, the trajectory may be adjusted in at least one coordinate direction. For example, the trajectory may be adjusted in a lateral direction. In such examples, the trajectory may also not be adjusted in a medial direction. In another example, the trajectory may be adjusted in both a lateral direction and a medial direction. It will be appreciated that in some embodiments the trajectory may be adjusted in any number of directions. The trajectory may also be adjusted to move based on the pattern of movement. In other words, the trajectory may move laterally upwards and downwards as the anatomical element moves.

The method 200 also comprises obtaining a second sample (step 220). The step 220 may be the same as or similar to the step 208.

In some embodiments, the step 220 may occur after a surgical step. In such embodiments, the step 220 may be used to confirm that that pattern of movement has not changed and/or to update the pattern of movement. For example, movement of the patient may affect a pattern of movement of an anatomical element and thus, a second sample may be desired to adjust the trajectory of the robotic arm based on the current breathing pattern of the patient. In another example, a shape of an anatomical element may change (whether due to drilling, cutting, or otherwise) and thus, the pattern of movement of the anatomical element may also change.

The method 200 also comprises determining a second pattern of movement (step 224). The step 224 may be the same as or similar to the step 212.

The method 200 also comprises adjusting the trajectory of a robotic arm (step 228). The step 228 may be the same as or similar to the step 216.

The present disclosure encompasses embodiments of the method 200 that comprise more or fewer steps than those described above, and/or one or more steps that are different than the steps described above.

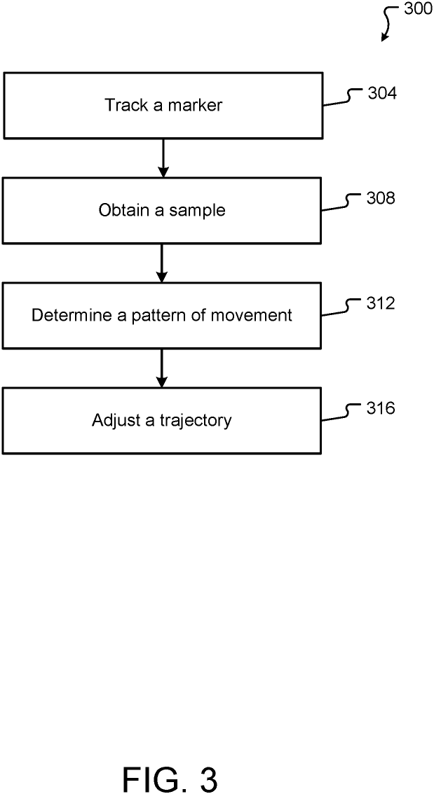

FIG. 3 depicts a method 300 that may be used, for example, for controlling a robotic arm.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in the memory and executed by the processor may cause the processor to execute one or more steps of a function as shown in method 300. One or more portions of a method 300 may be performed by the processor executing any of the contents of memory, such as a tracking 120, a signal processing 122, and/or a sample processing 124.

The method 300 comprises tracking a marker (step 304). The marker may be the same as or similar to the marker 136. In some embodiments, the marker may be coupled to a target anatomical element on which a surgical procedure is to be performed. For example, the surgical procedure may comprise inserting a surgical implant into a vertebra and the marker may be coupled to the vertebra. In other embodiments, the marker may be coupled to any portion of a patient. The tracking may be performed by a processor such as the processor 104 and/or a processor of a navigation system such as the navigation system 118 executing a tracking such as the tracking 120. The tracking may, for example, enable the processor to process image data (which may be received from, for example, an imaging device such as the imaging device 112 or an imaging device of a navigation system such as the navigation system 118) and/or sensor data (which may be received from, for example, a sensor such as the sensor 126) for the purpose of identifying the marker 136 and/or obtaining identifying information about the marker 136 from the image data or the sensor data. The information may comprise, for example, a plurality of poses of the marker. A sample of the plurality of poses of the marker for a time period may be obtained, which may correspond to a sample of a patient breathing pattern.

It will be appreciated that in some embodiments the patient breathing pattern may be controlled as described in the step 204 of the method 200 above.

The method 300 also comprises obtaining a sample (step 308). The step 308 may be the same as or similar to the step 208 of method 200 described above. In some embodiments, the sample may be obtained from the step 304 described above.

The method 300 also comprises determining a pattern of movement (step 312). The step 312 may be the same as or similar to the step 212 of method 200 described above.

The method 300 also comprises adjusting a trajectory (step 316). The step 316 may be the same as or similar to the step 216 of method 200 described above.

It will be appreciated that the method 300 may also comprise the steps 220, 224, and 228 of the method 200 described above. In other words, the method 300 may also comprise obtaining a second sample, determining a second pattern of movement, and adjusting the trajectory based on the second pattern of movement. Such steps may occur, for example, after a surgical procedure has been completed that may affect a position of the patient and thus, may affect the pattern of movement of the patient.

The present disclosure encompasses embodiments of the method 300 that comprise more or fewer steps than those described above, and/or one or more steps that are different than the steps described above.

As noted above, the present disclosure encompasses methods with fewer than all of the steps identified in FIGS. 2 and 3 (and the corresponding description of the methods 200 and 300), as well as methods that include additional steps beyond those identified in FIGS. 2 and 3 (and the corresponding description of the methods 200 and 300). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for controlling a robotic arm comprising:
a robotic arm;
a processor; and
a memory storing data for processing by the processor, the data, when processed, causes the processor to:
control a breathing pattern of a patient;
obtain a sample of the breathing pattern;
determine a pattern of movement of an anatomical element based on the sample; and
adjust a trajectory of the robotic arm based on the pattern of movement.

2. The system of claim 1, further comprising:
a marker disposed on the anatomical element and a navigation system configured to track the marker,
wherein the sample is obtained from the navigation system tracking a movement of the marker during a time period.

3. The system of claim 2, wherein the marker comprises at least one of an optical marker, an infrared light emitting diode, an electromagnetic marker, and an inertial measurement unit tracker.

4. The system of claim 2, wherein determining the pattern of movement of the anatomical element comprises determining a maximum height and a minimum height of the movement of the marker and a number of movements of the marker per the time period.

5. The system of claim 1, further comprising:
a ventilator configured to control the breathing pattern of the patient.

6. The system of claim 4, wherein the sample is obtained from the ventilator and the sample is based on the breathing pattern controlled by the ventilator.

7. The system of claim 6, wherein the sample is a waveform representing the breathing pattern, and wherein determining the pattern of movement comprises determining at least one of a crest, a trough, an amplitude, and a period of the waveform.

8. The system of claim 1, further comprising:
a sensor coupled to the robotic arm, the sensor configured to provide pose information of the robotic arm,
wherein the robotic arm is coupled to the anatomical element, and
wherein the sample is obtained from the sensor, the sample comprising a plurality of poses of the robotic arm for a time period.

9. The system of claim 8, wherein the plurality of poses corresponds to a movement of the anatomical element, and wherein determining the pattern of movement of the anatomical element comprises determining a maximum height and a minimum height of the movement and a number of movements of the robotic arm per the time period.

10. The system of claim 1, wherein the sample is a first sample and the pattern of movement is a first pattern of movement, and the memory stores further data for processing by the processor that, when processed, causes the processor to:
obtain a second sample of the breathing pattern;
determine a second pattern of movement of an anatomical element based on the sample; and
adjust the trajectory of the robotic arm based on the second pattern of movement when the second pattern of movement does not match the first pattern of movement.

11. The system of claim 1, wherein the trajectory is adjusted in at least one coordinate direction.

12. The system of claim 1, wherein the anatomical element comprises one or more vertebrae.

13. A system for controlling a robotic arm comprising:
a robotic arm coupled to an anatomical element;
a sensor coupled to the robotic arm, the sensor configured to provide pose information of the robotic arm;
a processor; and
a memory storing data for processing by the processor, the data, when processed, causes the processor to:
obtain a sample of a patient breathing pattern comprising a plurality of poses for a time period from the sensor;
determine a pattern of movement of the anatomical element based on the sample; and
adjust a trajectory of the robotic arm based on the pattern of movement.

14. The system of claim 13, wherein the plurality of poses corresponds to a movement of the anatomical element, and wherein determining the pattern of movement of the anatomical element comprises determining a maximum height and a minimum height of the movement and a number of movements of the robotic arm per the time period.

15. The system of claim 13, further comprising:
a ventilator configured to control the breathing pattern of the patient.

16. The system of claim 13, wherein the trajectory is adjusted in at least one coordinate direction.

17. A system for controlling a robotic arm comprising:
a marker disposed on an anatomical element;
a navigation system configured to track the marker;
a processor; and
a memory storing data for processing by the processor, the data, when processed, causes the processor to:
track the marker;
obtain a sample of a patient breathing pattern comprising a plurality of poses of the marker for a time period from the navigation system;
determine a pattern of movement of an anatomical element based on the sample; and
adjust a trajectory of the robotic arm based on the pattern of movement.

18. The system of claim 17, wherein the plurality of poses correspondent to a movement of the anatomical element, and wherein determining the pattern of movement of the anatomical element comprises determining a maximum height and a minimum height of the movement and a number of movements of the marker per the time period.

19. The system of claim 17, further comprising:
a ventilator configured to control the breathing pattern of the patient.

20. The system of claim 17, wherein the marker comprises at least one of an optical marker, an infrared light emitting diode, an electromagnetic marker, and an inertial measurement unit tracker.
